(19) United States
(12) Patent Application Publication
(10) Pub. No.: US 2010/0211051 A1
(43) Pub. Date: Aug. 19, 2010

(54) System and method for descemet's stripping automated endothelial keratoplasty (DSAEK) surgery

(76) Inventors: Philip Douglas Weston, Ledyard, CT (US); Donald Tan, Singapore (SG); Jod Mehta, Singapore (SG)

Correspondence Address: IRA S. DORMAN, 330 ROBERTS STREET, SUITE 200, EAST HARTFORD, CT, 06108, US

(21) Appl. No.: 12/657,943

(22) Filed: Jan. 28, 2010

Related U.S. Application Data
(63) Continuation-in-part of application No. PCT/GB2008/050943, filed on Oct. 16, 2008.

(51) Int. Cl. A61F 9/007

(52) U.S. Cl. 606/1

(57) ABSTRACT

The system for donor cornea implantation includes a preparation base having a well for receiving a posterior lamellar donor corneal lenticule, a cartridge disengageably mounted on the base adjacent the well, and a handle for disengageable attachment to a posterior end portion of the cartridge. In drawing the donor lenticule from the well into and through a bore or chamber of the cartridge, from the posterior end, the lenticule is caused to assume a double coil configuration. After attachment of the handle, removal of the assembly from the preparation base, and insertion of blade and adjacent body portions of the cartridge through an incision in the cornea, the coiled donor lenticule is pulled from the cartridge bore through its forward end, to uncoil automatically within the anterior chamber of the recipient's eye.

### CROSS-REFERENCE TO RELATED APPLICATION

This is a continuation-in-part of International Application No. PCT/GB2008/050943 entitled “Ophthalmic Surgical Device for Endothelial Keratoplasty for Descemet's Stripping Automated Endothelial Keratoplasty (DSAEK) Surgery,” filed 16 Oct. 2008 and published under International Publication No. WO 2009/050511, the entire specification of which is incorporated hereinto by reference thereto.

### BACKGROUND OF THE INVENTION

A paradigm shift in the approach to corneal transplantation is occurring, with new forms of anterior and posterior lamellar keratoplasty now enabling targeted replacement of only diseased layers of the cornea. These forms of lamellar corneal surgery are gradually replacing conventional full thickness penetrating keratoplasty (Tan D T, Mehta JS: “Future Directions in Lamellar Corneal Transplantation”; Cornea; in press).

Descemet's stripping automated endothelial keratoplasty (DSAEK) is a form of small incision and essentially sutureless surgery which represents the latest innovation in a series of posterior lamellar keratoplasty procedures that are now synonymous with the term “endothelial keratoplasty”. The DSAEK procedure involves stripping of diseased Descemet's membrane and endothelial cells through a small corneal incision, and replacement with a posterior lamellar donor corneal lenticule prepared with the use of the Automated Lamellar Therapeutic Keratoplasty (ALTK) unit (Price M O, Price F W Jr.: “Descemet's stripping with endothelial keratoplasty: comparative outcomes with microkeratome-dissected and manually dissected donor tissue”; Opthalmology; 2006 November; 113(10:1936-42).

With the adoption of any new surgical technique there is an inevitable learning curve for the surgeon and an accompanying evolution in techniques (see, for example: Price F W, Price M O: “Descemet's stripping with endothelial keratoplasty in 200 eyes: Early challenges and techniques to enhance donor adherence”; J Cataract Refract Surg. 2006; 32(3):411-8; Melles G R, Lander F, Beekhuis W H, Remeijer L, Binder PS: “Posterior lamellar keratoplasty for a case of pseudophakic bullous keratopathy”; Am J. Opthalmol. 1999 March; 127(3):340-1; Melles G R, Lander F, Nieuwendaal C: “Sutureless, posterior lamellar keratoplasty: a case report of a modified technique”; Cornea; 2002 Apr.; 21(3):325-7; Melles G R, Wijdh R H J, Nieuwendaal C P: “A technique to excise the Descemet membrane from a recipient cornea (descemetorhexis)”; Cornea; 2004 Apr.; 23(3):286-8; Terry M A, Ousley P J: “Replacing the endothelium without corneal surface incisions or sutures: the first United States clinical series using the deep lamellar endothelial keratoplasty procedure”; Opthalmology; 2003 April; 110:755-64; discussion 764).

One of the most challenging aspects of this procedure is the insertion of the donor posterior lenticule into the anterior chamber (AC) through a small incision, without inducing significant endothelial damage. The current widely performed technique requires insertion of the donor lenticule through a small 5 mm corneal or scleral incision by folding the lenticule and gripping the folded tissue with non-compressing forceps i.e. ‘taco insertion’. This traumatic handling of the donor has been criticized because of its propensity for damaging endothelial cells, with primary graft failure rates due to intraoperative endothelial cell loss and damage ranging from 6% to 45% in the current literature with this folding technique (Mearza A A, Qureshi M A, Rostron C K: “Experience and 12-month results of Descemet-stripping endothelial keratoplasty (DSEK) with a small-incision technique”; Cornea 2007 Apr.; 26(3):279-283). Damage to endothelial cells may occur as a consequence of mechanical folding of the donor, compression with holding forceps, and may also occur during intraocular manipulations to unfold the donor within the AC without the presence of an ophthalmic visco-surgical device (OVD). More recently, laboratory models of DSAEK have shown that folding of the donor lenticule for insertion into the AC and intraocular manipulation to unfold the donor is the stage most associated with significant endothelial cell loss (Lee W B, Sy H M, Holley G P, Edelhauser H F: “Descemet's Stripping Automated Endothelial Keratoplasty (DSAEK): Intra-Operative Effects on the Donor Corneal Endothelium”; IOVS supplement; 2007; abstract 1131). The endothelial damage is worse in the presence of associated anterior chamber shallowing.

Our own extensive in-vitro work has confirmed that significant endothelial damage occurs with the conventional folding technique, despite the use of commercially available ‘non-compression’ forceps (Goosey forceps, model no. 19090, Moria, Antony, France). Damage primarily occurring as a consequence of direct contact of folded endothelial surfaces where the folding forceps are applied, as well as along the folding crease (Mehta J S, Por Y M, Beuerman R W, Tan D T: “Glide Insertion Technique of Donor Cornea Lenticule during Descemet's Stripping Automated Endothelial Keratoplasty”; J Cat Refract Surg; in press). Our recent studies show that the mean endothelial cell loss is 39% with this technique, which is now described:

A 1 mm paracentesis is first made in the peripheral cornea opposite a 5 mm temporal scleral tunnel wound (for insertion of intraocular forceps). A standard, commercially available anterior chamber intraocular lens (TOL) Sheet's glide is trimmed to 4 mm in width along approximately half to ⅔ of its length. Using Kelman Macpherson forceps, the glide is inserted into the AC through the scleral tunnel, with the right hand, whilst a balanced saline solution (BSS) infusion is maintained on. The donor (both the anterior and posterior lamellae) is transferred to a Paton's spatula. A dispersive OVD is liberally applied over the endothelial surface particularly the peripheral circumference of the donor. Carefully gripping the posterior donor lamellar with Kawai intraocular capsulorhexis forceps (Asico) on the stromal side, the anterior cap is slid away from the spatula, ensuring that the posterior donor lamella stays on the spatula. OVD is placed on the anterior surface of the glide, and the Paton spatula with the posterior lenticule is carefully everted, corneal endothelial surface down, onto the OVD-covered portion of the glide. Holding the glide with the right hand with Kelman Macpherson forceps at its most posterior part, the left hand, passes the Kawai forceps through the paracentesis, across the AC and over the sheets glide, and is passed out through the scleral incision. The Kawai forceps is rotated, so that the forceps teeth are now obliquely or vertically aligned, and can be used to grasp the leading edge of the donor lamella, on the upper stromal surface. Once the forceps grasped the donor edge, the donor is rapidly pulled through the scleral incision in one steady, smooth motion until the donor is fully in the AC. At the same time, the glide was retracted out of the eye.

We have performed this technique in 24 cases of DSAEK surgery, with only one primary graft failure occurring (4.2%). This contrasts with our previous 20 cases using the folding technique which had primary graft failure rate of 25% (5 eyes). Our scanning electron microscope (SEM) studies confirm that significant reduction in endothelial loss occurs with this technique, with a mean cell loss of 9%, mostly occurring at the peripheral rim, which may be due to contact of the donor edges with the plastic sheets glide, despite the use of OVD, and some damage must still occur when the donor is dragged through the lips of the wound, as the donor endothelial surface is still potentially in contact with the inferior lip of the scleral wound. We have not encountered any cases of donor dislocation with this technique, although we have now seen one case of partial Descemet's detachment. Our only primary graft failure occurred during our first case using this technique and can be attributed to the use of an excessively thick donor lenticule (400 μm) which resulted in Descemet's detachment.

### BRIEF SUMMARY OF THE INVENTION

Accordingly, the broad objects of the invention are to provide an apparatus and method for inserting a corneal implant (also referred to variously as a “donor,” a “donor cornea,” a “corneal lenticule,” a “lenticule,” and a “donor corneal lenticule”) into the eye of a recipient without inducing significant endothelial damage.

A more specific object of the invention is to provide such an apparatus and method wherein and whereby the donor corneal lenticule is optimally temporarily deformed for effective insertion, while providing enhanced protection against significant endothelial damage before and during surgical implantation.

Additional objects of the invention are to provide an apparatus having the foregoing features and advantages which, in addition, is readily employed and is highly efficient and effective for its intended purposes; and to provide such a method which is readily carried out and is highly efficient and effective.

It has now been found that certain of the foregoing and related objects are attained by the provision, in accordance with a first aspect of one embodiment of the present invention, an assembly for inserting a corneal implant, the assembly comprising:

a deformation chamber for storing said implant in a pre-determined deformed shape;

said chamber comprising a housing member and a platform in sliding engagement with said housing member;

said platform having an insertion end for inserting into a corneal incision, with the housing member arranged to slide relative to said platform so as to at least partially enter said incision; and

biasing means for biasing the implant from the housing whilst in the deformed shape.

In a second aspect of the present invention, there is provided an assembly for resiliently deforming a corneal implant comprising:

a housing member having an internal curved surface, said housing member arranged to receive and hold said implant in a curved orientation, such that peripheral edges of said implant are directed away from said housing member;

a platform in sliding engagement with said housing member, said platform having a profiled face arranged to contact said implant on sliding said housing member into contact with said profiled face; wherein

said profiled face shaped to direct said peripheral edges towards said housing member on contact through said sliding engagement.

In a preferred aspect, the pre-determined shape may have a large radius of curvature, so as to prevent kinking or other subsequent damage to the implant.

In a further preferred aspect, the pre-determined shape may be any one of: a double coil, a cardioid, reverse curve, reverse double curve or corrugated.

In certain embodiments, the housing member may be an elongate curved member such as that shown as the donor chamber of FIGS. 1 to 4. Alternatively, the member may have a range of external shapes, whilst maintaining a curved inner shape for contact with said implant. The platform may be an elongate member such as that shown as the glide platform of FIGS. 1, 2 and 4.

In preferred embodiments, the biasing member may be forceps arranged to engage and pull the implant into place.

In certain instances the biasing means may include an element, such as a linearly directed projection, for pushing the implant into place. The element may take the form of a piston which fits in the deformation chamber or at least the housing, and which can be operated so as to push or eject the implant from the deformation chamber and into the eye of a recipient of the implant through the corneal incision. This embodiment is constructed in similar manner to a syringe. The element may alternatively take the form of a plate located at an end of a push-rod, the push-rod being connected to the plate at an edge thereof. This embodiment may be constructed in similar manner to a croupier's rake or croupier's tool. In both of these embodiments, the element may be held stationary relative to the recipient of the implant while the deformation chamber or at least the housing is moved backwards away from the incision, or the deformation chamber or at least the housing may be held stationary while the element is moved towards the incision.

In a further embodiment, the biasing means may engage a surface of the implant, through friction adhesion or other such means, and draw the implant from the assembly into place.

Embodiments of the present invention provide a disposable, plastic (or other appropriate surgical grade material, such as stainless steel) glide inserter which aims to simplify donor lenticule insertion in endothelial keratoplasty, and to reduce endothelial cell loss to below 5%.

Utilizing the glide concept enables insertion of the donor lenticule into the AC with no significant shallowing or loss of the AC, despite no OVD being present in the AC. An AC maintainer is preferably used to provide a constant flow of BSS to maintain the AC throughout the insertion procedure.

The glide prevents iris prolapse and also protects the endothelial surface of the donor from contact with iris tissue, and an AC IOL, if present. Insertion of the thin, flat glide results in minimal loss of BSS from the wound, and provides a stable, deep chamber at all times.

The novel device of certain embodiments, which can be an intrinsic part of the glide, is a transparent, closed chamber which is inserted into the wound with a complete seal, thus again providing a deep chamber with minimal BSS outflow.

Transparent plastic used in the device allows for clear visualization of the donor at all times.

In certain preferred embodiments, a fully closed chamber environment (consisting of the AC and the internal cavity of the glide) ensures full stability and minimal BSS flow, enabling the intraocular forceps to pull the donor into the AC in a controlled fashion with no risk of chamber collapse.

The internal diameter of the glide chamber may be calculated to allow for a donor lenticule of 9.5 mm in diameter with a thickness of up to 250 μm and in some cases up to 400 μm or greater to be coiled up and inserted with no endothelial contact with either endothelial surfaces, wound edges, or intraocular surfaces.

The donor can be double coiled in a novel fashion to ensure no contact between endothelial surfaces at any time during the insertion procedure.

Several sizes (small, medium, large) of the glide device may be provided to allow surgeon selection depending on wound size, donor diameter and donor thickness.

Embodiments of the present invention may be provided prepacked in a sterile package with a plastic (or other appropriate material, such as metal) preparation base which also functions as a base for coiling the donor.

The whole package may consist of two clear plastic components: a) Donor Chamber, b) Glide Platform, which come together to form a fully assembled Thumb Glide, and c) Preparation Base, which is useful for loading of the cornea within the Donor Chamber and for assembling the Thumb Glide of embodiments of the invention. Special glide forceps are a separate but also useful component of the procedure, but are not described in detail here.

The donor chamber or deformation chamber may be adapted so that it is sealable, for example by way of a screw cap or plug or stopper at one or both ends. In this way, a corneal implant can be prepared at a remote location, for example an eye bank, and stored the predetermined deformed shape in an appropriate nutrient solution or saline solution in a donor chamber or deformation chamber that is subsequently sealed. The sealed chamber can then be shipped to a surgeon in ready-to-use form. The surgeon then need only remove the seal(s) from the chamber before inserting the implant by way of the present invention.

Certain objects of the invention are attained by the provision, in accordance with the most preferred embodiments of the invention, of a cartridge for effecting coiling and insertion of a donor cornea implant, comprising a generally tubular portion including a sidewall defining a longitudinal bore, of curvilinear cross section, having open forward and rearward ends; a ridge element extending longitudinally along at least a portion of the bore and projecting inwardly thereinto from the sidewall and serving to induce curling of lateral edges of a donor corneal lenticule during longitudinal movement therealong; a glide or blade portion extending forwardly from the sidewall beyond the forward open end of the bore; and structure adjacent a rearward end of the tubular portion for disengageable attachment to a handle.

The cartridge will advantageously be integrally formed, as a single piece, and will desirably be molded from a substantially transparent or translucent synthetic plastic material. The ridge element provided will normally be formed with convexly curved lateral surfaces extending along its length and terminating in a common longitudinal apex. At the forward end of the tubular portion of the cartridge the sidewall will desirably be formed with a transaxial bevel that declines toward the blade portion, to facilitate physical access into the bore and insertion of the forwardmost part of the body portion into the AC of the recipient's eye. The curvilinear cross section of the bore will normally be generally circular or generally elliptical, and the bore will generally be of uniform cross-section along at least a major portion of its length.

Additional objects of the invention are attained by the provision of an assembly for effecting coiling and insertion of a donor cornea implant, comprised of a cartridge comprising a generally tubular portion including a sidewall defining a longitudinal bore, of curvilinear cross section, having open forward and rearward ends, a blade portion extending forwardly from the sidewall beyond the forward open end of the bore, and structure adjacent a rearward end of the tubular portion for disengageable attachment to a handle; and a handle disengageably attached to the cartridge, the handle comprising a gripping portion and means, adjacent one end, for disengageable attachment to the disengageable attachment structure of the cartridge, such that the handle enables facile manipulation of the attached cartridge.

The attachment means of the handle will normally engage the attachment structure of the cartridge in only a single orientation of relative rotation about a longitudinal axis, and in a snap-fit relationship. The handle will advantageously have closure structure at the attaching end, constructed to engage the tubular portion of the cartridge and to thereby to produce a liquid-tight seal of the open, rearward end of the tubular portion bore. The gripping portion of the handle will normally have opposite sides, with indicia on at least one of the opposite sides serving to distinguish it from the other side thereof (or it may be otherwise shaped to achieve the same purpose). Here again, the cartridge may additionally include a ridge element extending longitudinally along at least a portion of the bore and projecting inwardly thereinto from said sidewall, as described.

Objects of the invention are additionally attained by the provision of a system for use in preparing a donor cornea implant for delivery to an operating site and insertion into a recipient's eye, comprised of a cartridge and a handle, as hereinabove and hereinafter described, in combination with a preparation base comprised of a lower portion constructed for stable engagement with support means, and an upper portion. The upper portion of the preparation base defines a cornea-receiving recess or well for the receipt of a donor corneal lenticule, and has cartridge-receiving structure for removably engaging the lenticule-coiling cartridge in position with the open, rearward end of its bore proximate one side of the recess, for direct physical communication, and for allowing facile removal of the cartridge from the base. The upper portion of the base is also constructed to accommodate the handle, when it is assembled with an engaged cartridge, and either the cartridge or the preparation base, or both, have structure for inducing bilateral curling of a donor corneaa lenticule in the course of passing from the recess of the preparation base to the forward end of the cartridge bore.

In such a system the structure for inducing bilateral curling of a donor lenticule may, again, comprise a ridge element extending longitudinally along the bore of the cartridge, as described, or it may comprise a pair of laterally spaced shoulder elements, having curlinducing surfaces facing one another and disposed substantially at the intersection of the corneal lenticule-receiving recess and the cartridge-receiving structure (taking the form, for example, of curved surfaces forming a U-shaped throat section). Normally, the cartridge-receiving structure of the preparation base will coact slideably with structure of the cartridge in such manner that the cartridge can be received in only a single orientation of rotation about its longitudinal bore, and usually the cartridge-receiving structure and the attaching structure will permit receipt of the cartridge by relative movement in only one direction, and disengagement by relative movement only in the opposite direction. Objects of the invention are additionally attained by the provision of a method for effecting corneal implantation, which employs the assembly herein described. In accordance with the method, a donor corneal lenticule is placed, endothelial surface facing upwardly, in position adjacent the open rearward end of the longitudinal bore of the tubular portion of the cartridge; the donor lenticule is drawn into the bore, to reside therein, by force applied from the open forward end of the bore; the handle is attached to the cartridge using the means described; and the cartridge is so positioned, using the attached handle, as to enable the donor corneal lenticule to be drawn through the forward open end of the cartridge bore into the anterior chamber of recipient's eye.

Normally, the method of the invention will include the further steps of forming an opening (incision or paracentesis) into the anterior chamber of the cornea of a recipient, at least at generally diametrical locations; inserting the blade portion and the adjacent part of the body portion of the cartridge through one of the openings, inserting a gripping portion of an instrument through another opening so as to grip an edge of the donor corneal lenticule adjacent the open forward end of the cartridge bore; and pulling the lenticule into the anterior chamber of the recipient eye. Most desirably the method will include the additional step of causing the donor corneal lenticule to assume a double coil configuration, with no endothelial surface areas in mutual contact with one another, in the course of drawing the donor lenticule into the bore of the cartridge.

Throughout the description and claims of this specification, the words “comprise” and “contain” and variations of the words, for example “comprising” and “comprises,” means “including but not limited to,” and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

### BRIEF DESCRIPTION OF THE SEVERAL VIEWS OF THE DRAWINGS

For a better understanding of the present invention and to show how it may be carried into effect, reference shall now be made by way of example to the accompanying drawings, in which:

FIG. 1 is a plan view of a first embodiment;

FIG. 2 is a side elevation of the first embodiment;

FIG. 3 is a cross-sectional view through the first embodiment;

FIG. 4 is a perspective view of the first embodiment;

FIG. 5 is a side elevation showing the first embodiment mounted on a preparation base for loading of a donor lenticule;

FIG. 6 is a plan view of the preparation base of FIG. 5;

FIG. 7 shows a variation of the donor chamber with a croupier's rake biasing means;

FIG. 8 shows a variation of the donor chamber with a piston biasing means;

FIG. 9 shows a variation of the donor chamber with sealing caps or plugs;

FIG. 10 is a top perspective view of a second embodiment of the system of the invention;

FIG. 11 is a side elevational view of the system of FIG. 10 (the opposite side view being a mirror image thereof);

FIG. 12 is a plan view of the system of FIG. 10;

FIG. 13 is a front elevational view of the system;

FIG. 14 is a rear elevational view of the system;

FIG. 15 is a bottom perspective view showing the underside of the preparation base of which the system of FIG. 10 is comprised;

FIG. 16 is an exploded, perspective view showing the assembled handle and cartridge components comprising the system, displaced from the preparation base;

FIG. 17 is a perspective view of the handle and cartridge comprising the system, separated from one another and taken from the bottom (relative to the orientation in which corneal implant insertion is effected);

FIG. 18 is a bottom view of the handle and cartridge in assembly with one another;

FIG. 19 is a plan view of the assembly of FIG. 18;

FIG. 20 is a front view of the assembly, shown in the orientation of FIG. 17;

FIG. 21 is a sectional view of the assembly, taken along line 21-21 in FIG. 20;

FIG. 22 is a sectional view of the cartridge of the assembly, taken along line 22-22 in FIG. 21 and drawn to a greatly enlarged scale;

FIG. 23 is a sectional view of the preparation base of the system, taken along line 23-23 in FIG. 16 and drawn to an enlarged scale;

FIG. 24 is a fragmentary perspective view of the forward portion of the preparation base, depicting the placement of a donor corneal lenticule into a well formed in the top wall of the base;

FIG. 25 is a fragmentary perspective view of the forward portion of the preparation base with the cartridge component engaged thereon;

FIG. 26 is a view similar to FIG. 25, showing a donor corneal lenticule being drawn into the rear, or posterior, opening of the cartridge bore, or chamber;

FIG. 27 is a perspective view of the cartridge of the system, drawn to an enlarged scale and containing a donor corneal lenticule in a double coil configuration;

FIG. 28 is a cross sectional view taken along line 28-28 in FIG. 27, drawn to an enlarged scale and showing the donor corneal lenticule formed into a double coil configuration;

FIG. 29 is a perspective view showing the forward portion of the cartridge inserted through a scleral tunnel of a cornea, and also showing forceps introduced through a nasal paracentesis gripping the stromal leading edge of the donor and pulling it into the anterior chamber;

FIG. 30 is a perspective view similar to FIG. 29, showing the substantially uncoiled donor corneal lenticule being manipulated within the anterior chamber; and

FIG. 31 is another similar perspective view showing the donor corneal lenticule released and the cartridge and forceps removed.

### DETAILED DESCRIPTION OF THE INVENTION

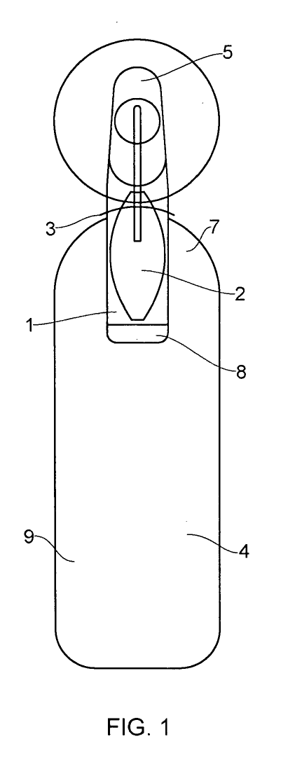

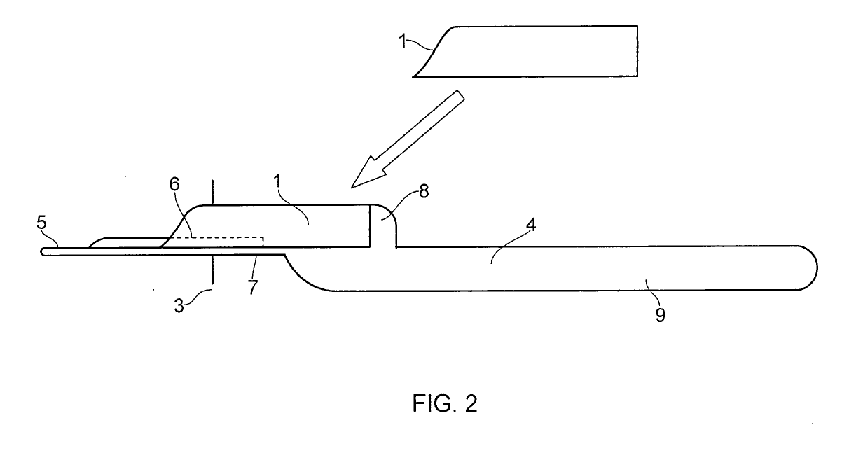

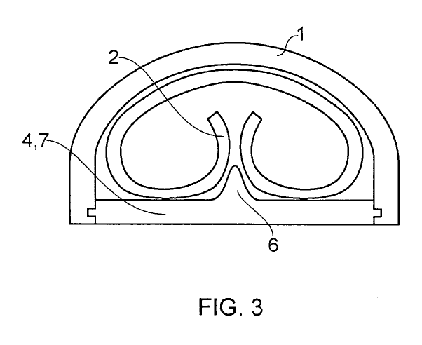

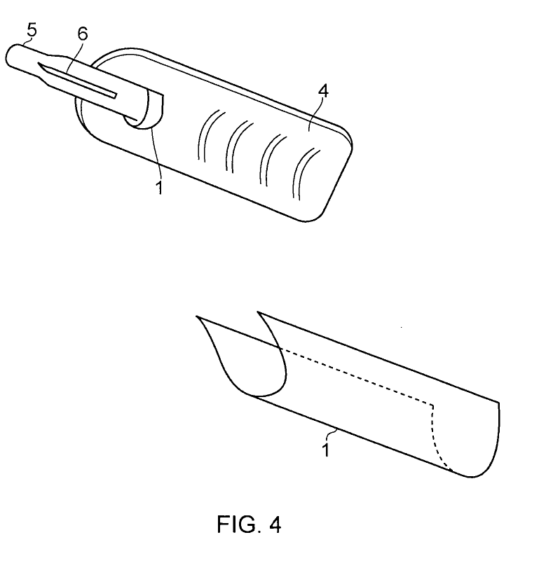

Turning in detail initially to FIGS. 1-4 and 7-9 of the drawings, the donor chamber 1 of a first embodiment is composed of clear plastic, and consists of a curved chamber, open at both ends. The donor lenticule 2 is dragged into the chamber and coiled up in a specific proprietary configuration, with the stromal surface snugly aligned to the inner surface of the chamber 1. The front end of the chamber 1 is bevelled in a cut-away design to allow ease of insertion through a scleral wound opening 3. The outer chamber diameter is designed to fit exactly through the scleral wound 3 with a semi-elliptical opening. The superior aspect of the anterior edge has a cut-away anterior surface to allow space for a pair of glide forceps (not shown) to enter the chamber 1 and grasp the leading stromal edge of the coiled donor 2. The posterior margins of the chamber 1 are straight-edged and grooved, to allow sliding of a glide platform 4 to close the chamber 1 inferiorly, with a glide member 5 protruding anteriorly (this will be inserted into the AC). The posterior back surface is also open, but will be closed by the posterior, matching back which is part of the glide platform 4.

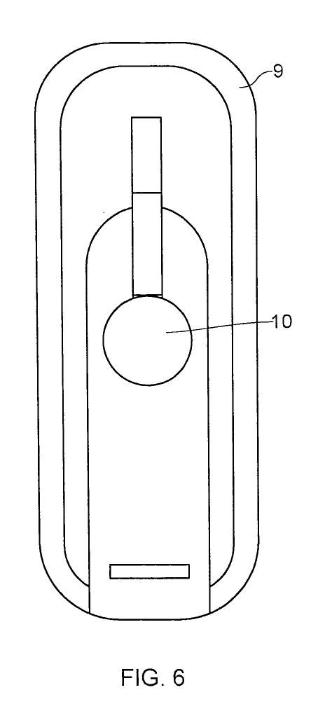

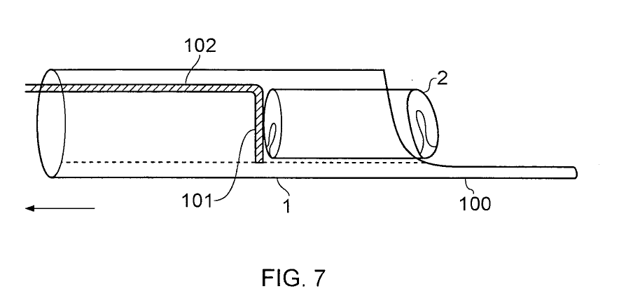

With particular reference to FIG. 7, there is shown a donor chamber 1 with a bevelled cut-away insertion end 100 and a coiled donor 2 located in the chamber 1. A biasing means in the form of a plate 101 on the end of a push-rod 102 is provided, the biasing means being similar in configuration to a croupier's rake. The biasing means can be used to push the donor 2 out of the chamber 1 into the recipient's eye when the insertion end 100 has been inserted through the scleral incision 3.

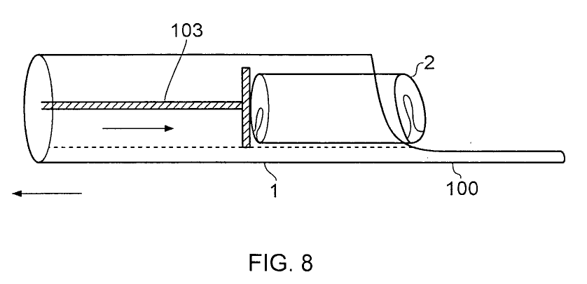

FIG. 8 shows a further variation in which the biasing means is configured as a piston 103.

In both the FIG. 7 and the FIG. 8 embodiments, the biasing means may be advanced while the chamber 1 is held stationary, or the biasing means may be held stationary while the chamber 1 is retracted.

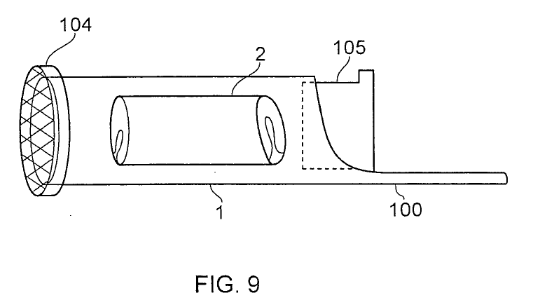

FIG. 9 shows a variation in which the donor chamber 1 is provided with a plug or seal 104, 105 at each end so as to allow the donor 2 to be stored in the chamber 1 in a suitable nutrient solution. In this way, the donor chamber 1 can be pre-filled with an appropriate donor 2 at an eye bank or the like, before being shipped to a surgeon in the form of a ready-to-use cartridge. This is particularly useful when the donor 2 requires special preparation after excision from a cadaver, since employees at the eye bank may be more skilled at such preparation than a surgeon, who will generally be more skilled at inserting the donor 2 into a recipient eye.

With further reference to FIGS. 1, 2 and 4, in particular, the glide platform 4 is composed of clear plastic, and has three sections. The anterior section 5 forms a flat glide portion which will be inserted into the AC. This is approximately 0.3 mm thick and slightly flexible. Mid-way along the glide surface, there is provided a 1.0 cm long central vertical protruding ridge 6 with curved margins present on the anterior aspect of the glide. The vertical ridge 6, which is 0.5 mm in height, enables the widest margins of the coiled donor 2 to coil up internally, stromal surface touching, hence preventing inadvertent endothelial surface touch which may occur if one edge of the donor 2 slides over the other. The middle portion 7 consists of a continuation of the anterior glide, which will form the base platform of the donor chamber 1, and the raised posterior back 8 of the chamber 1 (a back cowling) which attaches to the posterior aspect of the chamber 1, creating a closed chamber posteriorly. The posterior portion 9 consists of a broad base platform (approximately 2.5 cm thick) which may be flat or curved (concave surface upwards) and which will be the portion of the platform 4 which is grasped by the surgeon's thumb and forefinger during insertion. Ridges may be placed to avoid slippage. Threading the donor chamber 1 onto the glide platform 4 forms a complete thumb drive device, with a chamber holding the donor which will be completely coiled within.

A further design modification of the donor chamber 1 takes into account that the anterior opening of the donor chamber 1 will need to be inserted into the scleral wound 3 and introduction into the wound 3 with this large diameter bore may result in excessive outflow of chamber fluid and lead to temporary shallowing during introduction of the glide. The alternative design modification provides a thin, flexible, anterior cover flap over the donor chamber 1 opening, hinged superiorly. This will be an integral anterior extension of the upper surface of the chamber opening, and the flap may be inserted depressed down and tucked beneath the upper lip of the scleral wound 3, thus providing a temporary seal to the anterior opening of the donor chamber 1, thus reducing aqueous outflow and shallowing of the AC. Once the anterior margins of the donor chamber 1 have fully entered the scleral wound 3 and protrude into the AC, the flexible roof of this cover will spring upwards back into its original configuration, reopening the donor chamber 1 bore, thus allowing for the donor corneal lenticule 2 to be pulled unimpeded into the AC.

Where provided, glide forceps consist of a curved, single shaft intraocular titanium forceps designed to enter a 1 mm paracentesis passed through a nasal corneal opening opposite the temporal scleral incision 3. The forceps tip consists of a sharp-tipped opening and closing forceps design similar to other designs for intraocular capsulorhexis forceps but with a vertical orientation of the opening and closing teeth, as opposed to horizontal orientation in conventional capsulorhexis forceps. The forceps will be used to grasp the leading stromal edge of the coiled donor 2, while within the glide chamber, and used to pull the donor 2 through the donor chamber 1 into the AC.

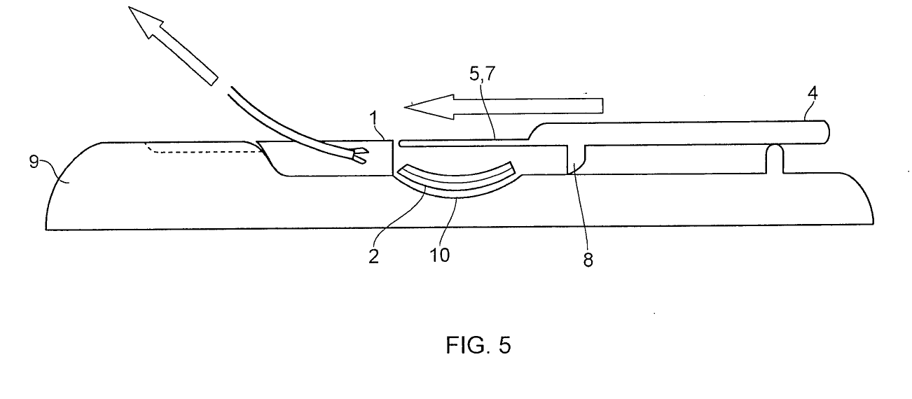

The preparation base 9, as shown in FIGS. 5 and 6, consists of plastic (or may be made of any other suitable material), and enables placement of the corneal donor 2 in position prior to coiled insertion into the donor chamber 1. The base 9 also enables stable positioning of the donor chamber 1 while the donor 2 is pulled into the chamber 1, and enables sliding closure of the glide platform 4 into the locked position on the donor chamber 1.

Stages of donor coiling, thumb glide assembly, insertion of the thumb glide through the scleral wound, donor pull-through, and removal of the thumb glide are described utilizing the apparatus of the foregoing figures:

1. The donor chamber 1 is placed onto the preparation base 9, upside down.
2. The donor corneal lenticule 2 (consisting of both the posterior lenticule and attached accompanying anterior cap) is placed, endothelial surface up, onto the circular platform 10 of the base 9. The donor 2 will have already been lamellar dissected and trephined to the desired diameter prior to placement, and liberally coated with dispersive OVD over the endothelial surface. One drop of BSS is placed on the circular platform 10 prior to donor placement to lubricate the surface and prevent the donor 2 sticking to the surface of the circular platform 10. Care should be taken to ensure that OVD is not present between the posterior donor stromal surface and does not overflow over the donor peripheral edges.
3. Careful loosening of the posterior donor lenticule from the anterior stromal cap is performed by gently separating the contacting surfaces with a BSS cannula, with injection of a small amount of BSS to lubricate the adjoining surfaces. This will allow easier separation of the posterior lenticule from the anterior cap.
4. The anterior leading stromal edge of the posterior lenticule is carefully marked with a surgical gentian violet marker pen so as to aid visualization of leading stromal edge.
5. The glide forceps is then used to grasp the inked leading stromal edge of the posterior lenticule, and is next used to carefully pull the donor 2 into the donor chamber 1, leaving the anterior cap behind, ensuring that there is full adherence of the stromal surface of the posterior donor to the inner margins of the donor chamber 1 (FIG. 5). One drop of BSS on the inner surface of the chamber 1 may reduce friction during this process. The curved surface of the chamber 1 will initiate concave coiling of the donor 2. If detachment or wrinkling of the donor 2 occurs during the initial pulling process, a second forceps can be use to guide the edges of the donor 2 to ensure that the donor coils accurately without wrinkling, is centered, and in direct contact with the inner edges of the chamber 1 at all times. The donor 2 is pulled all the way in into the chamber 1, until the leading edge of the donor 2 is approximately 0.5 mm from the anterior margin of the chamber 1. At this stage, the central outer edges of the donor lenticule will protrude vertically above the chamber sides.
6. The glide platform 4 is now be used to close the chamber 1. The leading glide edge of the glide platform 4 (which is introduced upside down) is threaded through the grooves of the chamber margins from the posterior margin and advanced slowly (FIG. 5). As the anterior flat portion of the glide encounters the vertically protruding central edges of the donor lenticule, careful further advancement will fold the donor edges inwards. Forceps may be used to tuck in the edges if necessary. When the edges then encounter the vertical protruding groove of the glide mid-way along insertion, the edges will be further coiled downwards, in a double coil configuration; the stromal surfaces will be in contact with each other and fold inwards more (this ensures that endothelial surfaces do not slide over each other). The glide will then be fully advanced until the posterior cap of the glide encounters the posterior margins of the donor chamber 1 and the glide is clipped into position over the chamber 1, ensuring a tight posterior seal. The complete thumb glide is now fully assembled, and can be fully detached from the preparation base 9, re-inverted upright, and set aside for insertion.
7. Recipient preparation: At this stage, the recipient cornea should already have been prepared (surface epithelium should already have been removed (if needed), trephination marking on the corneal surface performed, the scleral wound fashioned, nasal paracentesis performed, AC maintainer placed (either at superior or inferior limbus), and a peripheral iridectomy performed).
8. Descemet's stripping (if needed) is performed in the usual manner.
9. Introduction of the thumb glide containing the donor: With the surgeon holding the thumb glide with the thumb and forefinger (right hand for a right-handed surgeon), the anterior glide portion of the thumb glide is first inserted through the wound, with the AC maintainer on. The glide is advanced until the anterior edge of the donor chamber engages the superior lip of the scleral wound. The donor chamber edge is carefully slid beneath the superior lip, using forceps to lift the wound edge if necessary. The full process of insertion should be quick and deliberate, so as not to lose BSS and cause excessive chamber shallowing. The glide is then advanced fully through the wound 3, until the superior edge of the donor chamber 1 is just beyond the limbus, at which point the inked edge of the donor will be just visible through the peripheral clear cornea. At this stage, the glide will be fully engaged at the wound 3, which will be stretched open by the chamber diameter. Elasticity of the sclera will enable the glide to be fully pressed against the wound opening, ensuring a tight seal with minimal leakage of BSS. The AC should be fully maintained and deep throughout this procedure, with the AC maintainer on, and minimal egress of BSS will ensure that the donor remains stable within the donor chamber 1 at this point.
10. The glide forceps is introduced through the nasal paracentesis opening, with the surgeon's left hand, and advanced across the AC above the glide. The forceps tip is passed just beneath and beyond the donor chamber 1 and the teeth opened to grasp the leading stromal edge of the donor 2 which can be clearly visualized by the ink marking. The forceps is then gently retracted out of the AC, pulling the donor 2 smoothly into the AC. As the donor 2 enters the AC, it will unfold by itself in the deep chamber. If incomplete folding of the outer edges occurs, the forceps may be used to gently agitate the donor sideways back and forth, to facilitate full unfolding. The forceps can also be used to position the donor 2 centrally in alignment with the superficial trephination on the recipient corneal surface. At this point, the grip on the donor is not yet released.
11. Once the donor 2 is fully unfolded, and in position, the donor glide should be smoothly but quickly retracted completely out of the wound, allowing the scleral wound 3 to close. Some egress of BSS will be encountered during this manoeuvre, but BSS inflow from the AC maintainer will rapidly refill and deepen the AC. Continual holding onto the donor with the forceps will ensure that the donor does not slip out of the wound. If the slightly stretched scleral wound remains partially open, an assistant may help to close the scleral wound 3. Once the glide is fully retracted and the AC fully reformed, a small air bubble may be injected into the AC, beneath the donor, by the right hand of the surgeon with a 30 gauge needle through a separate limbal site—this will float the donor up against the posterior corneal surface, ensuring that the donor does not settle downwards onto the iris or IOL surface. Finally, the donor 2 may now be fully released from the forceps. Completion of the DSAEK procedure by suturing the wound and paracentesis sites, followed by a full air injection for donor adherence, can now be performed and the procedure completed in the usual manner.

One alternative thumb glide insertion technique entails modification of the glide to enable near-complete insertion of the entire glide (containing the donor) into the AC, thereby positioning the coiled corneal lenticule safely in a controlled manner within the centre of the AC. The lenticule is then much closer to the opposite chamber angle, enabling a shorter glide forceps to be used. The glide is then simply retracted out of the AC, leaving the donor in position to uncoil of its own accord.

There are several novel and unique concepts of embodiments and techniques of and involving the present invention, which are listed below. The list is not exhaustive, and the advantages may be obtained individually or in any appropriate combination.

1. Glide concept—coupled with the use of an AC maintainer on throughout the procedure, a deep chamber is maintained at all times, the glide prevents iris prolapse out of the wound and prevents contact of the endothelial surface of the donor with iris or IOL surfaces.
2. Close chamber concept of donor insertion—the closed chamber of the thumb glide ensures no turbulent outflow of BSS during insertion, and a stable, deep and quiet chamber. The chamber of the glide acts as an extension of the AC, allowing for safe and simple donor entry into the AC.
3. The clear plastic design of the glide allows for full visualization of the donor and AC details at all time, facilitating accurate grasping of the donor.
4. The disposable nature of the glide ensures no risk of prion disease and other microbial and biofilm contamination inherent in non-disposable alternatives which need cleaning and sterilization.
5. The double coiling principle of the donor within the glide chamber allows for minimal endothelial contact with stroma or adjacent endothelial surfaces, thus reducing endothelial damage. The circular coiling of the donor also avoids sharp folding creases of the donor, again limiting endothelial cell damage. The double coiling shape also minimizes that total diameter of the glide, thus enabling the smallest wound size to be achieved with no donor touch, coupled with an ideal elliptical profile of the internal diameter of the glide. Various different glide sizes (three, for example) enable the surgeon to select the appropriate glide size according to the desired wound size, donor diameter, and thickness of the donor lenticule.
6. The donor is completely protected from contact with the wound edges during insertion, by the fact that the walls of the glide abut the entire wound margin, thus further reducing endothelial damage during insertion.

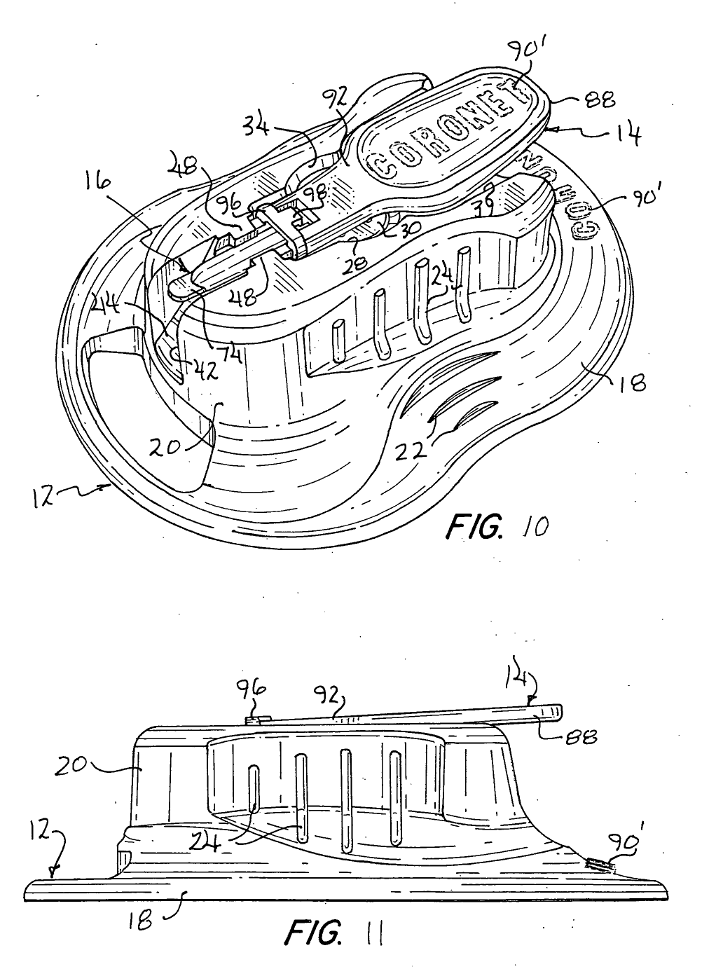

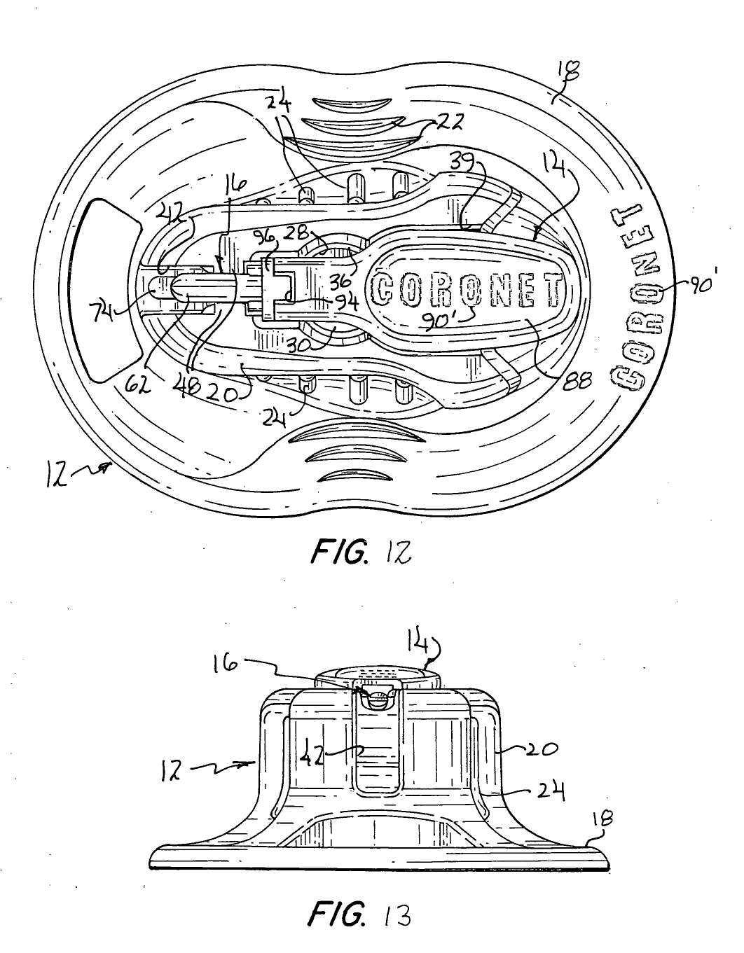

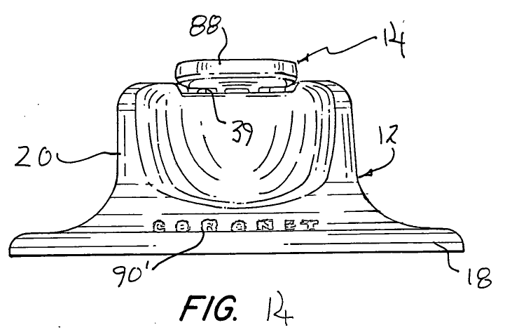

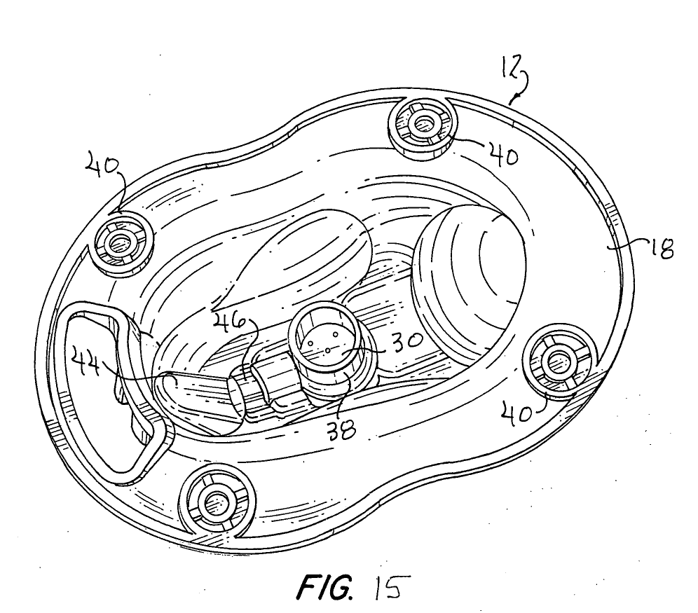

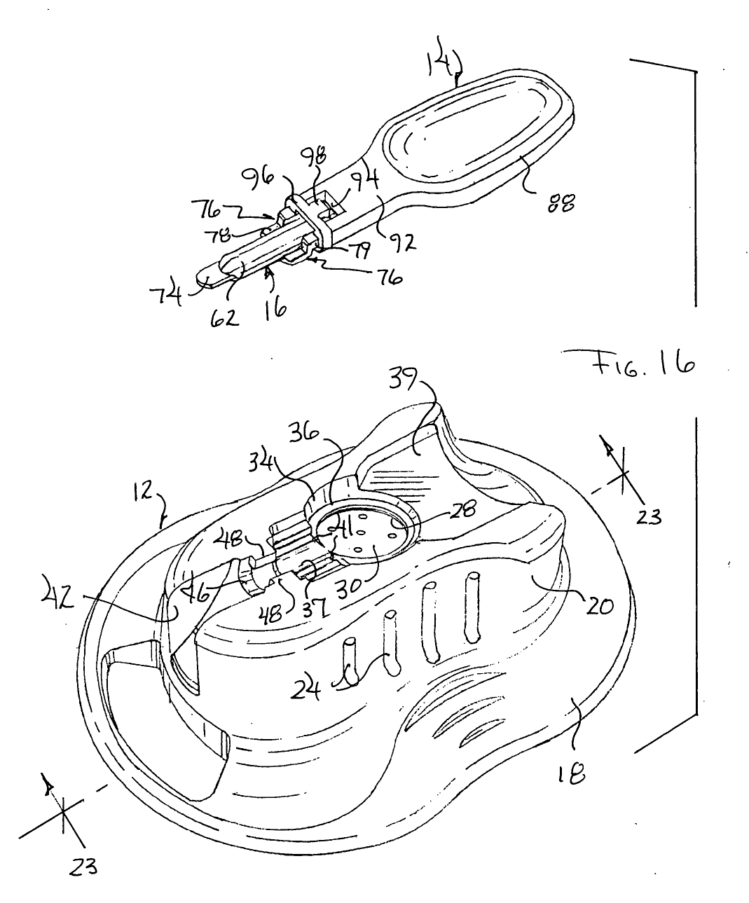

Turning now in detail to FIGS. 10 through 31 of the drawings, therein illustrated is a second, and most preferred, embodiment of the system of the invention, which comprises a preparation base, a handle, and a cartridge, generally designating respectively by the numerals 12, 14, and 16. Each of these components will desirably be molded from a suitable synthetic material (that is, plastic).

The preparation base 12 is symmetrical about a longitudinal centerline, and includes a bottom flange portion 18 and an upstanding pedestal portion 20, each of which is provided with gripping elements 22, 24, respectively. It will be noted that the flange portion 18 has the word CORONET, designated 90′, molded into it so as to the project above the adjacent surface; apart from its literal significance, the structure forming the word provides texture to further facilitate secure gripping of the base.

A donor-receiving recess or well 28, formed in the top wall of the pedestal portion 20 of the preparation base 12, is defined by a perforated bottom wall 30, a low, generally circular and partially surrounding wall element 32, a partially surrounding, generally conical section 34, and an annular surface 36 between the element 32 and the section 34, the element 32, the section 34 and the surface 36 being interrupted at diametrical positions. A relatively wide trough 39, of arcuate cross section, is also formed in the top wall of the pedestal portion 20 and extends rearwardly from the well 28.

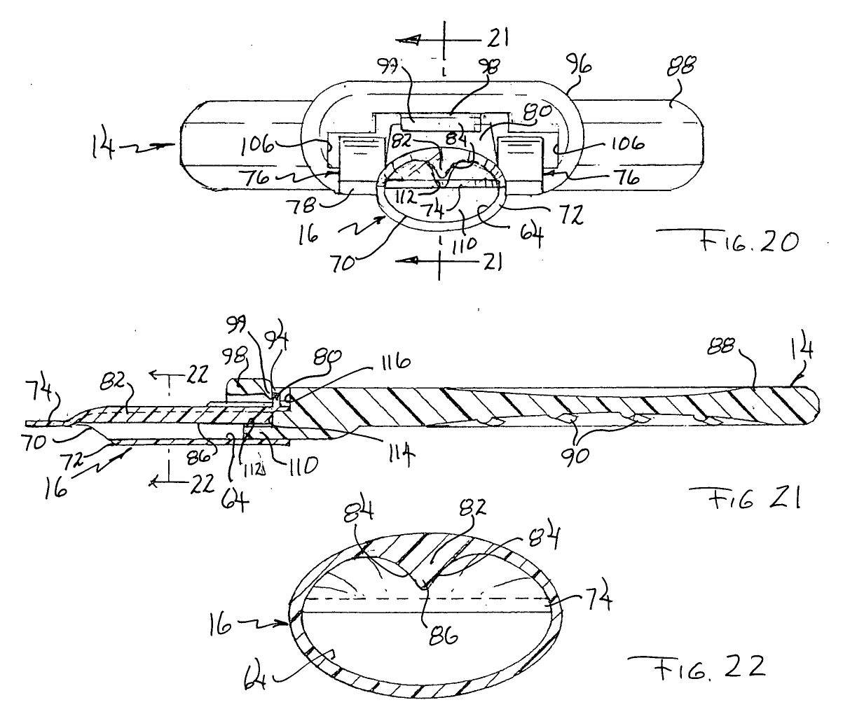

A pair of lugs 48 extend laterally inwardly toward one another at the top of the pedestal portion 20, and define the upper margins of a passage 37 between the well 28 and the channel 42. The surfaces of opposing shoulder elements 41, at the intersection of the well 28 and the passage 37, are of curvilinear form and (as is perhaps best seen in FIGS. 23 and 25) define a generally U-shaped throat or entrance to the passage 37. The shoulder elements 41 function to induce upward curling of the opposite lateral edges of the donor corneal lenticule upon entrance into the cartridge bore.

A sloping wall 44 defines the bottom of a channel 42 formed at the front of the pedestal portion of the preparation base 12 and terminating rearwardly in an arcuate wall portion 46. Foot elements 40 are formed in the underside of the bottom flange portion 18, and a nipple 38, for attachment of a hose or other conduit to enable evacuation of the well 28, extends downwardly from its bottom wall 30.

The cartridge 16 will normally be molded from a transparent to translucent plastic material, which allows the surgeon a clear view when drawing the implant into the cartridge and subsequently pulling it out. The sidewall forming the tubular body 62 of the cartridge defines a longitudinal bore or chamber 64, preferably of generally elliptical cross section, extending between an open posterior end 66 and open anterior end 70 surrounded, respectively, by a generally annular end surface 68 and a beveled end surface 72.

A glide member 74 (in the form of a blade or tongue-like element) extends forwardly from the anterior end 70 of the sidewall of the body 62 and is of generally planar form (oriented, in a crosswise direction, parallel to the major elliptical axis of the chamber 64). Stepped flanges, generally designated by the numeral 76, are formed along the opposite sides of the cartridge body 62, each flange 76 consisting of an anterior element 78 and a posterior element 79, the end faces of which posterior elements 79 are coplanar with the generally annular surface 68 surrounding the posterior end opening 66. A catch element 80 is formed at the posterior end of the cartridge 16, between the flange elements 79, and provides means for disengageably securing the cartridge 16 to the handle 14 when the components are properly assembled with one another, as will be described presently.

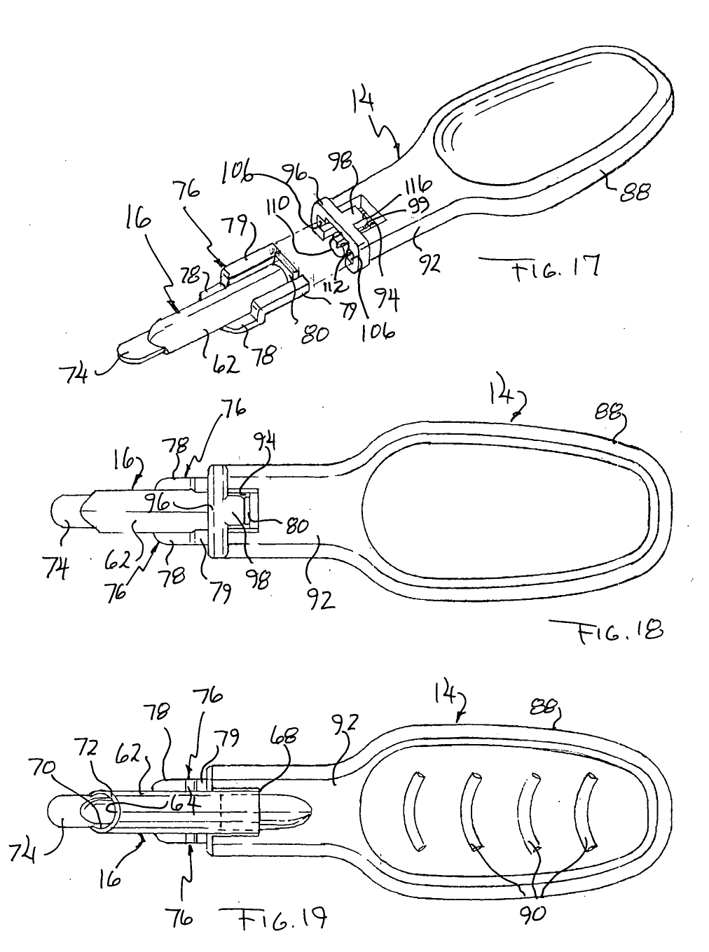

As best seen in FIGS. 21, 22, and 28, an elongate ridge or protrusion 82 extends longitudinally along most of the length of the body 62 of the cartridge 16 and projects into the bore or chamber 64. The ridge 82 has convexly curved lateral bearing surfaces 84, which extend lengthwise along its opposite sides and terminate in a common, rounded apex 86. The ridge 82 may serve the following functions: as a guide, to promote and maintain the double coil configuration, in ratios of confronting portions of from about 50:50 through to 60:40; as a shield between the two stromal surfaces, reducing the risk of tissue adhesion between those surfaces; when wet, as a lubricative surface, reducing friction between the two coils and, in turn, reducing the internal stress on the tissue cells; and, perhaps most importantly, as an anatomical support to naturally hold the tissue in a way that removes the prospect of its collapse (i.e., to crumple) on itself when the assembly has been inverted (i.e., with the ridge on the bottom).

The handle component 14 of the system includes a relatively broad posterior portion 88 on which is formed (on the side that faces upwardly in the orientation of cornea implantation) raised rib elements 90, and a narrower anterior portion 92 into which is formed an axially extending indentation 94; as seen in FIGS. 10 and 12, the opposite side of the handle may carry raised lettering 90′, again to afford a better grip. A collar element 96 partially surrounds the end of the anterior portion 92, and is formed with a small prong 98 having an underlying tab element 99 across an inner edge. A pair of parallel slots 106 extend inwardly along the opposite sides of indentation 94, and serve to slidably receive the posterior flange elements 79 on the cartridge body 62; it will be appreciated that the cartridge 16 can be assembled with the handle 14 in only one orientation of rotation about its longitudinal axis. When the cartridge 16 is fully engaged with the handle 14, a finger 110 is disposed within the bore 64 of the cartridge 16, with an apical portion 86 of the ridge 82 seated in the groove 112 along the finger 110. A first wall portion 114 near the inner end of the slot bears upon the proximate end of the ridge 82, and a second wall portion 116 bears upon the confronting portion of the annular surface 68 at the posterior end of the cartridge bore 64. This structure produces an effective liquid-tight seal at the posterior end of the cartridge, which is held in assembly therewith due to the snap-fit engagement of the tab 99 on the prong 98 of the handle collar 96 in front of the catch 80 on the cartridge 16 (the tab having a bevel to facilitate assembly, and the prong 98 being sufficiently resiliently deflectable to enable disassembly, as appropriate).

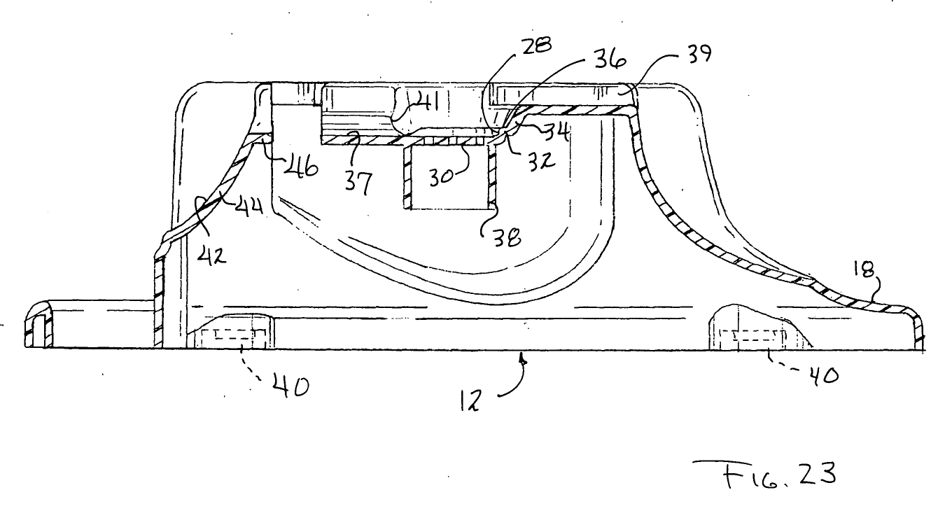

Use of the system of FIGS. 10 through 23 will be evident from the foregoing description, particularly taken with reference to FIGS. 24 through 31. As depicted in FIG. 24, a donor corneal lenticule “D” is initially placed into the well 28, with the endothelial surface facing upwardly, using a Paton's spatula “S” and forceps “F”. A cartridge 16 is then assembled on the pedestal portion 20 of the preparation base 12, with the anterior flange elements 78 slidably engaged within the grooves 43 that are formed under the lugs 48. It will be appreciated that this structure permits the cartridge to be received in only a single orientation of rotation about its longitudinal axis, and by pushing it forwardly from a position over the well 28; removal requires movement of the cartridge, relative to the base, in the opposite direction.

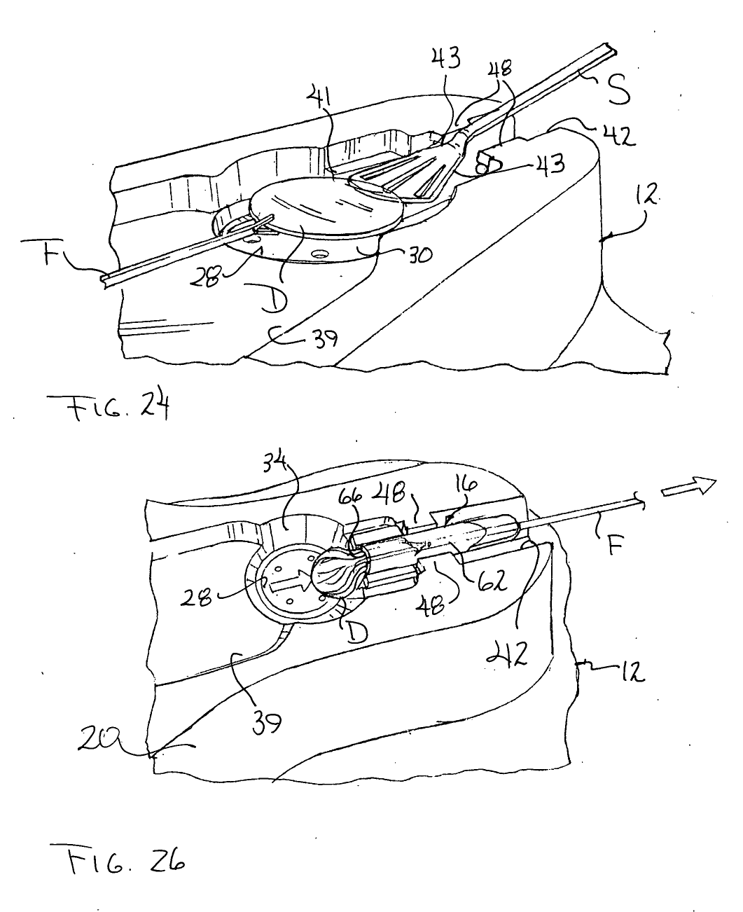

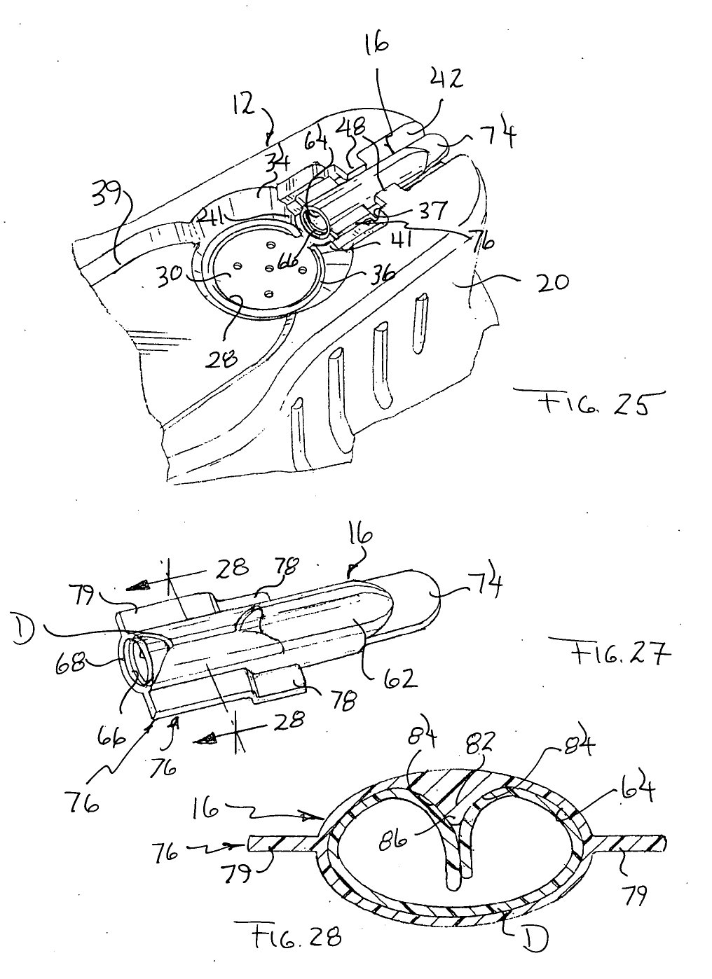

The donor corneal lenticule “D” is then drawn through the posterior end opening 66 of the body 62 of the cartridge 16 and into its bore 64, using forceps “F” introduced through the anterior opening 70 to grip the stromal edge and apply a pulling force thereupon, as indicated by the open arrows in FIG. 26. In the course of drawing the lenticule “D” into the bore 64, its lateral edges are curled inwardly, by contact with the surfaces of shoulder elements 41 and with the bearing surfaces 84 of the ridge 82 (albeit either feature may suffice), ultimately to assume a double coil configuration, as shown in FIG. 27 and (particularly) FIG. 28, wherein there is seen to be no contact between any endothelial areas of the donor corneal lenticule; it is also seen in FIG. 28 that the donor corneal lenticule may be asymmetric (i.e., have offset confronting portions) in its double coil configuration.

When the donor “D” is drawn fully into the chamber 64 (as depicted in FIG. 27), the handle 14 is attached to the cartridge 16, with the finger 110 inserted into the bore 64 and the mating proximate surface elements in mutual contact, so as to effectively seal the bore, and with the tab 99 of the prong 98 engaged behind the catch element 80 so as to secure the assembly. It will be appreciated that, as depicted in for example FIGS. 10 through 14, the handle cartridge assembly is inverted relative to the orientation in which the donor “D” will be introduced for implantation.

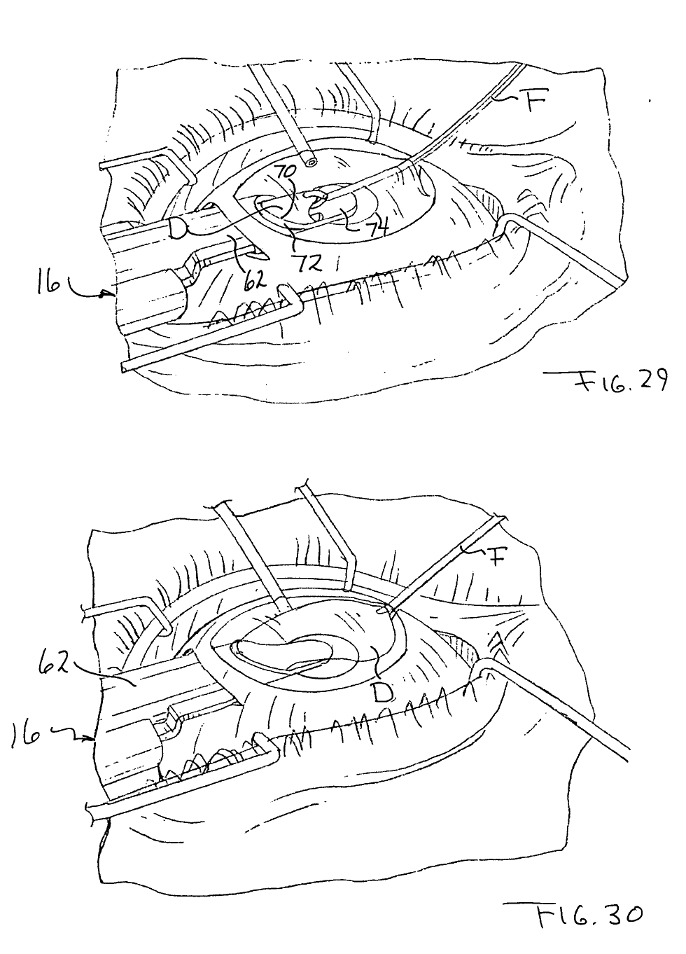

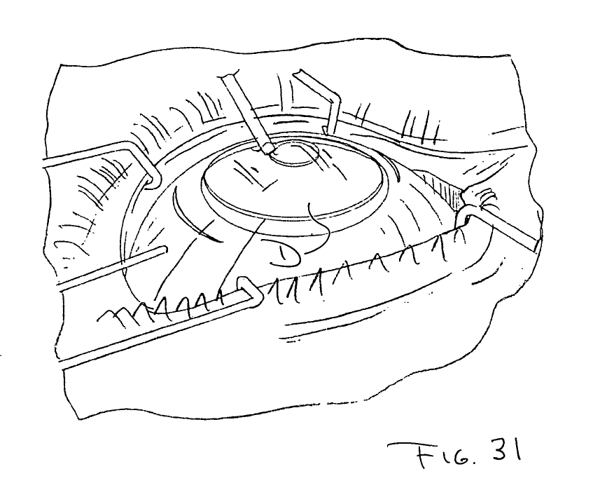

After the donor corneal lenticule D is loaded into the cartridge, the assembly is removed from the preparation base 12 (by a rearwardly sliding action), turned right-side up and, in the initial phase of implantation, the blade element 74 and adjacent cartridge body portion are introduced through the scleral tunnel of the anterior chamber so as to form a complete seal of the wound; this phase is depicted in FIG. 29. Subsequently (as seen in FIGS. 29 and 30), the donor “D” is pulled from the bore 64 through the anterior end opening 70 of the cartridge body 62 and into the anterior eye chamber, again using forceps “F” introduced through a diametrically located opening (nasal paracentesis); the glide member 74 is thereafter withdrawn (by retracting the cartridge). While still holding the implant with the forceps, an air bubble is injected beneath the implant, using a hypodermic needle or a small cannula that is inserted through the incision from which the glide 74 has been removed. The forceps are then released and withdrawn, and (as seen in FIG. 31) the implant floats upwardly in the anterior chamber and adheres to the posterior surface of the recipient's cornea (from which diseased endothelial tissue has been removed). The incisions are then closed in the usual manner.

Thus, it can be see that the present invention provides an apparatus and method for inserting a posterior lamellar donor corneal lenticule into the eye of a recipient, while providing enhanced protection for the endothelial cells both before and also during surgical implantation and without inducing significant endothelial damage. Using the apparatus and method of the invention, the donor lenticule is temporarily deformed in a manner that is optimal for effective insertion and for affording a high degree of endothelial cell protection. The lenticule may, in certain embodiments, be pushed into the anterior chamber using appropriate biasing means, but it is found that withdrawing the lenticule from the cartridge with a pulling action, using forceps inserted through a small incision into the anterior chamber on a side opposed to that through which the glide member of the cartridge is inserted, causes significantly less endothelial damage. The apparatus is readily employed, and is highly efficient and effective for its intended purposes; the method is readily carried out, and is also highly efficient and effective.

As indicated above, corresponding opposite lateral portions of a donor corneal lenticule, formed into a double coil configuration, need not be in mutual registration; indeed, optimal results may be realized when, for example, the coiled portions of such a deformed lenticule exhibit a ratio of asymmetry as great as about 60:40. Desired asymmetry may be produced by the surgeon through relative lateral displacement of the forceps within the cartridge when the donor lenticule is first drawn thereinto from the well in the preparation base.

Having thus described thus invention, what is claimed is:

1. A cartridge for effecting coiling and insertion of a donor cornea implant, comprising a generally tubular portion including a sidewall defining a longitudinal bore, of curvilinear cross section, having open forward and rearward ends; a ridge element extending longitudinally along at least a portion of said bore and projecting inwardly thereinto from said sidewall, said ridge element being constructed to induce inward curling of opposite lateral edges of a donor corneal lenticule during movement therealong; a blade portion extending forwardly from said sidewall beyond said forward open end of said bore; and structure adjacent a rearward end of said tubular portion for disengageable attachment to a handle.

2. The cartridge of claim 1 wherein said cartridge is integrally formed as a single piece.

3. The cartridge of claim 2 wherein said cartridge is molded from a substantially transparent or translucent synthetic plastic material.

4. The cartridge of claim 1 wherein said ridge element is formed with convexly curved lateral surfaces extending along its length and terminating in a common longitudinal apex.

5. The cartridge of claim 1 wherein, at the forward end of said tubular portion, said sidewall is formed with a bevel that declines toward said blade portion to facilitate physical access into said bore and insertion into the anterior chamber of a recipient's eye.

6. The cartridge of claim 1 wherein said curvilinear cross section is generally circular or generally elliptical.

7. The cartridge of claim 6 wherein said bore is of uniform cross-section along at least a major portion of its length.

8. An assembly for effecting coiling and insertion of a donor cornea implant, comprised of:
a cartridge comprising a generally tubular portion including a sidewall defining a longitudinal bore, of curvilinear cross section, having open forward and rearward ends; a blade portion extending forwardly from said sidewall beyond said forward open end of said bore; and structure adjacent a rearward end of said tubular portion for disengageable attachment to a handle; and
a handle disengageably attached to said cartridge, said handle comprising a gripping portion and means, adjacent one end, for disengageable attachment to said disengageable attachment structure of said cartridge, said handle enabling facile manipulation of said cartridge.

9. The assembly of claim 8 wherein said attachment means of said handle engages said attachment structure of said cartridge in only a single orientation of relative rotation about a longitudinal axis.

10. The assembly of claim 8 wherein said attachment means of said handle engages attachment structure of said cartridge in a snap-fit relationship.

11. The assembly of claim 8 wherein said handle has closure structure at said one end thereof, constructed to engage said tubular portion of said cartridge and to thereby produce a liquid-tight seal of said normally open rearward end of said bore of said tubular portion.

12. The assembly of claim 8 wherein said gripping portion of said handle has opposite sides, and wherein indicia are provided on at least one of said opposite sides of said gripping portion to distinguish it from the other side thereof.

13. The assembly of claim 8 wherein said cartridge additionally includes a ridge element extending longitudinally along at least a portion of said bore and projecting inwardly thereinto from said sidewall, said ridge element being constructed to induce inward curling of opposite lateral edges of a donor corneal lenticule during longitudinal movement therealong.

14. A system for use in preparing a donor cornea implant for delivery to an operating site and insertion into a recipient's eye, comprised of:
a cartridge comprising a generally tubular portion including a sidewall defining a longitudinal bore, of curvilinear cross section, having open forward and rearward ends; a blade portion extending forwardly from said sidewall beyond said forward open end of said bore; and attaching structure adjacent a rearward end of said tubular portion for disengageable attachment to a handle;
a handle having a gripping portion and means adjacent one end for disengageable attachment to said disengageable attachment structure of said cartridge, said handle, when attached, enabling facile manipulation of said cartridge; and
a preparation base comprised of a lower portion constructed for stable engagement with support structure, and an upper portion, said upper portion defining a lenticule-receiving recess for the receipt of a donor corneal lenticule and having cartridge-receiving structure for removably engaging said cartridge in position with said open rearward end of said longitudinal bore thereof proximate one side of said recess and for direct physical communication therewith, and for allowing facile removal of said cartridge from said base, said upper portion of said base being constructed to accommodate said handle when it is assembled with said engaged cartridge, at least one of said cartridge and said preparation base having structure for inducing inward curling of opposite lateral edges of a donor corneal lenticule in the course of passing from said lenticule-receiving recess of said preparation base to said forward end of said bore of said cartridge.

15. The system of claim 14 wherein said structure for inducing inward curling comprises a ridge element extending longitudinally along at least a portion of said bore of said cartridge and projecting inwardly thereinto from said sidewall.

16. The system of claim 14 wherein said structure for inducing inward curling comprises a pair of laterally spaced shoulder elements disposed substantially at the intersection of said lenticule-receiving recess and said cartridge-receiving structure.

17. The system of claim 14 wherein said cartridge-receiving structure of said base coacts with structure of said cartridge in such manner that said cartridge can be received in only a single orientation of rotation about said longitudinal bore thereof.

18. The system of claim 17 wherein said cartridge-receiving structure of said base and said attaching structure of said cartridge coact to permit receipt of said cartridge by movement of said cartridge only one direction, relative to said preparation base, and to permit removal of said cartridge by relative movement only in the opposite direction.

19. The system of claim 18 wherein said cartridge is slideably engaged by said cartridge-receiving structure of said preparation base.

20. A method for effecting corneal implantation, said method employing the assembly of claim 8 and comprising the steps:
(1) supporting a donor corneal lenticule, endothelial surface facing upwardly, in position adjacent the open rearward end of said longitudinal bore of said tubular portion of said cartridge;
(2) drawing said donor corneal lenticule into said bore, to reside therein, by force applied from said open forward end of said bore;
(3) attaching said handle to said cartridge using said means for disengageable attachment and said disengageable attachment structure of said cartridge; and
(4) utilizing said handle to so position said cartridge as to enable said donor corneal lenticule to be drawn through said forward open end into the anterior chamber of recipient's eye.

21. The method of claim 20 including the further steps of forming openings in the cornea and into the anterior chamber of a recipient's eye, at least at generally diametrical locations; inserting said blade portion and an adjacent body portion of said cartridge through one of said openings; inserting a gripping portion of an instrument through the other of said openings; using said gripping portion of the instrument to grip an edge of said donor corneal lenticule adjacent said open forward end of said cartridge bore; and pulling said donor corneal lenticule into the anterior chamber of the recipient's eye.

22. The method of claim 20 including the further step of causing said donor corneal lenticule to assume a double coil configuration, with no endothelial surface areas in mutual contact with one another, in the course of drawing said donor corneal lenticule into and through said bore of said cartridge.

23. The method of claim 22 wherein, in said further step of causing said lenticule to assume a double coil configuration, confronting portions of said donor corneal lenticule are caused to lie in an asymmetric relationship to one another.
